(19) United States
(12) Patent Application Publication
(10) Pub. No.: US 2019/0254532 A1
(43) Pub. Date: Aug. 22, 2019

(54) BABY LANGUAGE TRANSLATION SYSTEM AND METHOD OF USING THE SAME

(71) Applicant: Margaret T. Shaw, Prescott, AZ (US)

(72) Inventor: Margaret T. Shaw, Prescott, AZ (US)

(21) Appl. No.: 16/278,386

(22) Filed: Feb. 18, 2019

Related U.S. Application Data
(60) Provisional application No. 62/633,216, filed on Feb. 21, 2018.

(51) Int. Cl. A61B 5/0205; A61B 5/00; A61B 5/11; A61B 5/16; G10L 25/66

(52) U.S. Cl. CPC A61B 5/0205; A61B 5/0002; A61B 5/11; A61B 5/742; A61B 5/02405; A61B 5/165; A61B 5/7267; G10L 25/66; A61B 5/7405

(57) ABSTRACT

Provided is a baby language translation system and method including a database comprising at least one software program, a computer device configured to receive at least one audio cue from an infant and analyze the audio cue using the at least one software program, and at least one output device. The computer device is configured to recognize the at least one audio cue and use the at least one software program to translate the at least one audio cue and output a translation of the at least one audio cue to the at least one output. The disclosed baby language translation system may further include a recording device configured to record at least one of a type of biometric data. The recording device is further configured to record the at least one audio cue from the infant and transmit the audio cue to the computer device.

### CROSS-REFERENCE TO RELATED APPLICATIONS

The present application is related to and claims priority from prior provisional application Ser. No. 62/633,216, filed on Feb. 21, 2018, entitled “BABY LANGUAGE TRANSLATION SYSTEM,” the contents of all of which are incorporated herein by reference and are not admitted to be prior art with respect to the presently claimed invention via the mention in this cross-reference section.

### FIELD OF THE INVENTION

The present invention relates generally to deciphering the internal and external factors influencing infant physiological and psychological regulation, and more particularly to a system and method for translating the internal and external factors into meaningful information to assist caregivers in attending to the needs of preverbal dependents.

### BACKGROUND

A baby has no words but information can be deciphered by observing reflex sounds, body movements, temperature, and physiological process to express some psychological condition. For example, the baby laughs when it is in good humor and cries when it has some uncomfortable condition. Also, the baby may be hot which, when combined with a specific audible noise and/or movement, might indicate sickness or anxiety. Because the baby communicates by noise or movement, it is up to the person caring for the baby to determine what the baby needs at that moment, and to meet that need. Parents and caregivers often would like to know why their baby is crying and what they can do to resolve the crying.

Accordingly, there exists a need to provide a way for new parents, and caregivers tending to newborns and other infants, to determine accurately and easily the reason why their baby is crying.

### SUMMARY

An embodiment of this disclosure provides a baby language translation system including a database comprising at least one software program and a computer device configured to receive at least one audio cue from an infant and analyze the audio cue using the at least one software program. The system further includes at least one output device configured to connect wirelessly to the computer device. The system database is configured to store a known data set and the at least one software program comprises an algorithm which interacts with the database. The disclosed computer device is configured to recognize the at least one audio cue and use the at least one software program to translate the at least one audio cue and output a translation of the at least one audio cue to the at least one output device. In an embodiment, the disclosed baby language translation system may further include a data sensing device comprising an audio sensing device and a recording device, wherein the recording device is configured to record at least one of a type of biometric data, wherein the biometric data comprises at least one of a respiration rate (RR), a heart rate variability (HRV), an ambient temperature (TEMP), electrodermal activity (EDA) or a movement feature. In various embodiments, the recording device is further configured to record the at least one audio cue from the infant and transmit the audio cue to the computer device. The output device may comprise a display configured to provide useful auditory or visual output based upon the translation of the at least one audio cue.

Another embodiment of this disclosure provides a baby language translation system comprising at least one software or database programmed to receive a translated or converted audio cue and analyze the received translated or converted audio cue based on an algorithm which uses a known data set. The at least one software or database of the disclosed system outputs at least one of a response, command, or information in response to receiving and analyzing the translated or converted audio cue, wherein the at least one of the response, command, or information diagnoses or otherwise provides useful information with respect to the translated or converted audio cue. Various embodiments may further include a recording device or an audio sensing device configured to receive an audio cue of a baby, and at least one output device wirelessly connected to a computer device which is electronically connected to the at least one software or database and to the recording device or the audio sensing device. In an embodiment, the algorithm of the disclosed system may combine auditory data from categorical Dunstan Baby Language (DBL) classification data with biometric data including at least one of respiratory rate (RR), heart rate variability (HRV), ambient temperature (TEMP), electrodermal activity (EDA), and movement to provide an analysis of a received audio cue.

An embodiment of the disclosure provides a method for translating baby language into clear communicable word forms, the method comprising recording, using at least one data sensing device, an audio cue and transmitting, electronically, the audio cue to at least one computer device. The method may further include converting, using the at least one computer device, the audio cue to a converted audio cue comprising at least one of an electronic message or a non-propagating signal, and analyzing, using a database electronically connected to the at least one computer device, the converted audio cue, making an analyzed audio cue comprising at least one of an analyzed electronic message or an analyzed non-propagating signal. An embodiment further includes recording, using the at least one data sensing device, at least one biometric value corresponding to the audio cue to aid in at least one of the converting of the audio cue and the analyzing of the converted audio cue. Next, the method provides automatically categorizing, using the database, the analyzed audio cue into a categorized audio cue and then, outputting, using the at least one computer device, at least one directive to an output device, wherein the directive corresponds to the categorized audio cue.

### BRIEF DESCRIPTION OF THE DRAWINGS

Having thus described examples of the disclosure in general terms, reference will now be made to the accompanying drawings, which are not necessarily drawn to scale, and wherein like reference characters designate the same or similar parts throughout the views. The particular objects and features of the instant disclosure as well as the advantages related hereto will become apparent from the following description taken in connection with the accompanying drawings, and wherein:

FIG. 1 is an illustration of a baby language translation system according to an embodiment of the disclosure;

FIG. 2 is an illustration of a method for translating baby language into clear communicable word forms according to an embodiment of the disclosure.

### DETAILED DESCRIPTION

The following description of the disclosed embodiments of this disclosure is intended to enable someone skilled in the prior art to make and use that which is disclosed, but is not intended to limit the claims to these particular exemplary embodiments.

### INTRODUCTION

The responsiveness of a caregiver to an infant's signals is responsible for supporting how infants regulate their emotional systems. Research shows that child physiological dysregulation is a causal precursor to psychological dysregulation. Caregiver responses that do not accurately solve an infant's needs may undermine emotional regulation development at a time when the most critical neural development of regulatory mechanisms occurs, the first years of life.

Research has been done on infant crying that has demonstrated associations between characteristics of the volume, tone, and frequency of the crying. Until recently, communication was thought of as consisting mostly of language, and since babies do not talk, some consider them incapable of communication. However, baby noises, such as crying, contain linguistically salient aspects of human speech that are physiologically based and adapted for communication. Human speech is divided into linguistic and paralinguistic, or suprasegmental, aspects. The linguistic, or lexical, components refer to the elements, which develop meaning, as phonemes become syllables and words to be organized into phrases and sentences by rules of syntax. Qualitative aspects of speech, the intonation patterns, inflection, stress, intensity, and general melody form, constitute the paralinguistic component. These so called “prosodic” features of speech have their acoustical correlates in the timing (duration), amplitude (intensity) and fundamental frequency (dominant pitch) of phonation. It is these features that convey attitudes and emotional states from the baby to their parent or caregiver. Communication relies heavily on these prosodic features of speech. They are the first aspects of language to appear in the vocal behavior of the human infant, the cry. Thus, infant noises and crying are part of the matrix for later language development.

These very specific preverbal vocalizations are the result of reflexes stemming from the 10^{th} cranial nerve or vagal nerve complex in the autonomic nervous system. The vagal nerve complex is comprised of the dorsal and ventral vagal nerves. The dorsal vagal nerve complex (DVC) provides primary control of subdiaphragmatic visceral organs, such as the digestive tract. The ventral vagal nerve complex (VVC) provides primary control of supradiaphragmatic visceral organs, such as the esophagus, bronchi, pharynx, and larynx. The VVC also exerts important influence on the heart. In order to maintain homeostasis, the central nervous system responds constantly, via neural feedback, to environmental cues. Stressful events disrupt the rhythmic structure of autonomic states and subsequently result in reflex sounds that are created from the body attempting to regulate the nervous system. These reflexes exist in all humans from the moment we are born and continue to assist our regulatory functions throughout life. Since the VVC plays such an integral role in the nervous system it follows that the heart rate, electrodermal activity, and respiration variability correlated with reflex vocal cues is a reliable index of nervous system activity, meaning we will be able to correlate objectively observable sounds, temperature, heart rate, electrodermal activity, movement, and other nervous system cues to reach a reasonable conclusion about what the infant needs.

Physiological dysregulation can be associated with early psychological experiences. Studies show a connection between emotional dysregulation at 5 and 10 months, and parent-reported problems with anger and distress at 18 months. Low levels of emotional regulation behaviors at 5 months were also related to non-compliant behaviors at 30 months. Smoking, self-harm, eating disorders, and addiction have all been associated with early childhood emotional or physiological dysregulation. Somatoform disorders may be caused by a decreased ability to regulate and experience emotions or an inability to express emotions in a positive way. Emotional dysregulation is also found in people who are at increased risk to develop a mental disorder, in particular an affective disorder such as depression or bipolar disorder, attention deficit hyperactivity disorder, borderline personality disorder, narcissistic personality disorder, and complex post-traumatic stress disorder. Emotional dysregulation is also found among those with autism spectrum disorders.

Research suggests that based on a caregiver's actual history of providing the appropriate care based on the infant's cues, the infant constructs an Internal Working Model (IWM) of self that will subsequently guide the infant's behavior and expectations of attachment figures, most significantly in times of stress for the duration of their lives.

Nonetheless, the foregoing observations, and the research underlying them, have not thus far led to any means by which a parent, or even a pediatrician or a specialist in linguistics, physiology or psychology, could reliably distinguish what an infant wants, from the sounds made by the infant in crying.

As an initial matter, the terms “baby” and “infant” are used interchangeably throughout this document. As one of ordinary skill in the relevant art would know, the Dunstan Baby Language (DBL) teaches that an infant having between 0 to 3 months of age makes known sound reflexes, also referred to herein as vocalizations. According to the teachings of the DBL, after an infant matures past approximately 3 months of vocalization, an infant usually begins developing more advanced sounds beyond the sound reflexes or known 5 vocalizations.

While the DBL sound reflexes appear to be most easily discernable for infants having between 0 to 3 months old, various factors (e.g., premature birth) may affect the maturity of a baby's vocal chords or other related anatomy, or various factors may otherwise cause an infant to make discernable sound reflexes up to 6 months old or perhaps even 9 months old. Thus, when used herein in relation to various embodiments of the instant disclosure, an “infant” or “baby” may be defined as a child having between 0 to 3 months of age. According to various other embodiments of the instant disclosure, an “infant” or “baby” may be defined as a child having between 0 to 6 months of age. According to still other various embodiments of the instant disclosure, an “infant” or “baby” may be defined as a child having between 0 to 9 months of age.

Moreover, and as discussed in detail below, the algorithm of the instant disclosure, in conjunction with a known data set, is configured to provide a diagnosis or useful information with respect to an audio signal received from an infant. Further, various embodiments of the disclosed baby language translation system comprising the disclosed algorithm and being configured to provide a diagnosis or useful information regarding an infant's audio signal, is a system that will be helpful for understanding infants that are not only between the ages of 0 to 3 months, but older as well.

The present invention overcomes the problems cited above by providing a baby language translation system comprising sensing components, data capture, translation software, and remote transmission to communicate information to a user in real time.

It is an object of the present invention to provide a system and method of translating biometrics and noises made by infants to determine the infant's needs. The system comprises an audio recording device for receiving an audio cue made by the infant; at least one database on at least one computer device, wherein the computer device is preferably configured to receive the audio signal from the audio recording device and analyze the audio signal based on an algorithm; and at least one output device configured to connect wirelessly to the at least one computer device.

The at least one database is preferably configured and arranged to store a known data set of vocalizations and biometric data related to their meanings. The at least one computer device is preferably configured and arranged to recognize at least one audio cue and use the algorithm within the software program to translate the at least one audio cue and output the translation and/or directive to the at least one portable device.

The system will preferably further comprise an at least one sensor configured to record at least one type of biometric data, wherein the biometric data is further comprised of a respiration rate, at least one heart rate, at least one ambient temperature, at least one electrodermal activity, at least one baby internal temperature, at least one movement feature, or at least one location system.

The method of translating baby language into clear communicable word forms comprising the steps of: recording at least one subtle vocal cue; interpreting said subtle vocal cues; analyzing said subtle vocal cues; and outputting at least one directive to a portable device.

The method for translating baby language into clear communicable word forms, wherein recording at least one subtle vocal cue further comprises a step of recording at least one biometric value to aid in interpreting and analyzing said at least one subtle vocal cue.

The present invention is a baby language translation system preferably comprising at least one database further comprising at least one software program; at least one computer device, wherein the at least one computer device is configured to receive at least one audio cue and analyze the at least one audio cue using the at least one software program; and at least one output device configured to connect wirelessly to the at least one computer device. The at least one database is preferably configured to store a known data set. The at least one software program is preferably further comprised of an algorithm configured to interact with the at least one database. The at least one computer device is preferably configured and arranged to recognize the at least one audio cue and use the at least one software program to translate the at least one audio cue and output the translation to the at least one output device.

The known data set stored on the at least one database is preferably comprised of various vocalizations along with their known meanings. The five “words” of the infant “universal pro-language” were transliterated by Dunstan as: “Neh”=hungry; “Eh”=need to burp (upper gas); “Oah (Owh)”=tired (sleepy); “Eairh (Eargghh or Eair)”=stomach cramp (lower gas); “Heh”=physical discomfort at skin level (for example, feeling hot or wet).

The algorithm is preferably configured to convert the at least one audio cue to an audio signal and analyze the audio signal using the at least one database on the at least one computer device to provide a translation of the audio cue based on comparing the at least one audio cue to the list of known vocalizations. The at least one software program or at least one database outputs a response, command and/or information in response to such received converted audio signal that diagnoses or otherwise provides useful information with respect to such audio signal.

The baby language translation system preferably further comprises a recording or listening device configured for recording the at least one audio cue. The recording or listening device preferably picks up the audio cue emitted by the infant as an audio signal and electronically transmits the audio signal to the at least one computer device. The at least one computer device uses the known data set of vocalizations and definitions to analyze and interpret the audio signal and generates a translation based on the known data set using the at least one software program.

The at least one computer device preferably transmits the translation to the at least one output device preferably configured to provide useful auditory or visual output based on the recorded or heard audio cue. The useful auditory or visual output is preferably a combination of the distress of the infant and a suggested instruction for solving the infant's distress.

The baby language translation system preferably further comprises at least one sensor preferably configured to record at least one type of biometric data. The biometric data is preferably further comprised of at least one respiration rate, at least one heart rate, at least one electrodermal activity, at least one ambient temperature, at least one baby dermal temperature, and/or at least one movement feature. The at least one type of biometric data is sent to the at least one computer device to utilize in aiding the translation process.

Research has indicated that the known vocalizations are also attached to a plurality of distinct biometric data to more accurately reflect the baby's communication of a particular condition. The plurality of biometric data includes various cues that correlate to the vocalizations as follows: NEH=tongue on the roof of mouth/shortened breath; OWH=longer exhale and short inhales with mouth in the shape of O; EH=chest tightens short inhale and exhale; EAIR=increased distress increased respiration and heart rate; and HEH=increased distress increased respiration and heart rate.

The known data set would preferably further comprise the plurality of biometric data defined above. The at least one sensor would preferably be combined with the audio recording device to capture various biometric data of the baby at the same time as the audio cue. The at least one sensor is preferably configured to record at least one respiration rate, at least one heart rate, at least one electrodermal activity, at least one ambient temperature, at least one baby internal temperature, or at least one movement feature.

A method for translating baby language into clear communicable word forms comprising the steps of; recording or hearing at least one audio cue; interpreting said subtle vocal cues, or converting said at least one audio cue to an electronic message, signal, or the like; analyzing said at least one audio cue and/or said electronic message or signal and automatically categorizing said at least one audio cue; and outputting at least one directive to an electronic device. The recording at least one audio cue further comprises a step of recording at least one biometric value to aid in interpreting and analyzing said at least one audio cue.

Although the present invention has been described by way of example, it should be appreciated that variations and modifications may be made without departing from the scope of the invention. Furthermore, where known equivalents exist to specific features, such equivalents are incorporated as if specifically referred to in this specification.

In an embodiment, a baby language translation system comprises at least one database comprising an at least one software program; at least one computer device, wherein said computer device is configured to receive at least one audio cue and analyze said audio cue using said at least one software program; and at least one output device configured to connect wirelessly to said at least one computer device; wherein said at least one database is configured to store a known data set; wherein said at least one software program is comprised of an algorithm configured to interact with said at least one database; and wherein said at least one computer device is configured and arranged to recognize at least one audio cue and use said at least one software program to translate said at least one audio cue and output said translation to said at least one output device.

In another embodiment, the above described baby language translation system further comprises at least one sensor configured to record at least one type of biometric data, wherein said biometric data is further comprised of a respiration rate, at least one heart rate, at least one electrodermal activity, at least one ambient temperature, at least one baby internal temperature, or at least one movement feature.

In another embodiment, the above described baby language translation system further comprises a recording or listening device configured for recording at least one audio cue and transmitting said audio cue to said at least one computer device.

In another embodiment, the above described baby language translation system further comprises the at least one output device is configured to provide useful auditory or visual output based upon said translation of said audio cue.

In an embodiment, a method for translating baby language into clear communicable word forms comprises the steps of recording or hearing at least one audio cue; transmitting said at least one audio cue to said at least one computer device; interpreting said at least one audio cue, or converting said at least one audio cue to an electronic message, signal, or the like; analyzing said at least one audio cue and/or said electronic message or signal and automatically categorizing said at least one audio cue; and outputting at least one directive to an electronic device based upon the analyzed at least one audio cue.

In another embodiment, a method for translating baby language into clear communicable word forms as described above, further comprises the step of recording or hearing at least audio cue further comprising a step of recording at least one biometric value to aid in interpreting and analyzing said at least one audio cue.

In an embodiment, a baby language translation product or system comprises an algorithm and known data set running on a computer system designed to receive translated audio or other information input from a listening device; and further comprises at least one output device configured to connect wirelessly to and interact with such algorithm and known data set via computer software or code; wherein said at least one database or algorithm is configured to recognize at least one audio cue and output said translation and/or diagnostic/suggested behavior information or output.

In another embodiment, a baby language translation system comprised of at least one software or database programmed to receive a translated or converted audio signal and analyze said converted audio signal based on an algorithm and/or known data set; and wherein said software or database outputs a response, command and/or information in response to such received converted audio signal that diagnoses or otherwise provides useful information with respect to such audio signal.

In an embodiment of the baby language translation system described above, the system further comprises a recording or listening device configured for receiving an audio signal of a baby.

In another embodiment of the baby language translation system described above, the system further comprises at least one output device configured to connect to or communicate with said at least one computer device and provide useful auditory or visual output based upon the recorded or heard audio cues.

In another embodiment of the baby language translation system described above, the system further comprises at least one sensor configured to record at least one type of biometric data, wherein said biometric data is further comprised of a respiration rate, at least one heart rate, at least one electrodermal activity, at least one ambient temperature, at least one baby internal temperature, or at least one movement feature.

In an embodiment of the baby language translation system described above, the biometric data may further include at least one baby “dermal” temperature. A baby's dermal temperature is defined as a temperature surrounding a baby as measured between a baby's skin and a first layer of clothing or covering. In various embodiments, a covering may be a blanket, sheet or other such fabric or material used for regulating an infant's temperature.

### System Examples

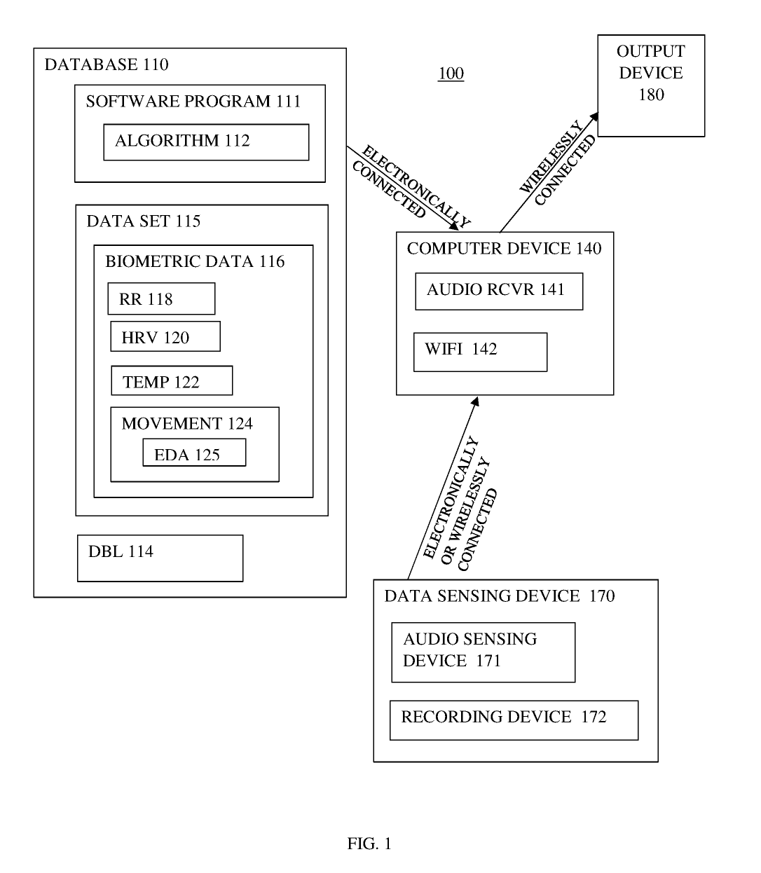

FIG. 1 is an illustration of a baby language translation system according to an embodiment of the disclosure. As shown in FIG. 1, an embodiment of the instant disclosure is a baby language translation system (100) having a database (110) that includes at least one software program (111). The system may further include a computer device (140) comprising an audio receiver (141) and wireless communication (WIFI) capability (142). The computer device (140) is configured to receive at least one audio cue from an infant or another user via the audio receiver (141) and analyze the audio cue using the at least one software program (111). In various embodiments, the at least one software program (111) comprises an algorithm (112) configured to interact with the database (110).

The system may also have at least one output device (180) which connects wirelessly or electronically to the computer device (140). The computer device (140) receives the at least one audio cue and uses the software program (111) and the algorithm (112) therein to translate the audio cue and output a translation of the at least one audio cue to the at least one output device (180). The output device (180) may comprise a display configured to provide useful auditory and/or visual output based upon the translation of the at least one audio cue.

In an embodiment, the database (110) of the system is configured to store a known data set (115) which includes biometric data (116). The biometric data (116) comprises at least one of a respiration rate (RR)(118), a heart rate variation (HRV)(120), an electrodermal activity (EDA)(125). an ambient temperature (TEMP)(122), or movement (124). In an embodiment, a movement (124) may be an electrodermal activity (EDA)(125). In another embodiment, the biometric data (116) may further include an internal temperature of an infant or baby.

In various embodiments, the baby language translation system (100) may have a data sensing device (170) including an audio sensing device (171) and a recording device (172). The recording device records at least one type of biometric data from an infant or user, including at least one of RR, HRV, EDA, TEMP, or a movement feature. In an embodiment, the recording device (172) may be configured to record the at least one audio cue from the infant or user and transmit the audio cue to the computer device.

In various embodiments, the algorithm (112) combines auditory data from categorical Dunstan Baby Language (DBL) classification data (114) with biometric data (116) utilizing a machine learning model. The software or database outputs at least one of a directive, response, command, or information in response to receiving and analyzing the translated or converted audio cue. The response, command, or information subsequently diagnoses or otherwise provides useful information with respect to the translated or converted audio cue which the computer device sends to the output device or display.

In various embodiments, the algorithm may further include data that is tracked and stored by the baby language translation system and used by the algorithm for reference. For example, tracked and stored data might include temporal information related to when a baby last ate, slept, urinated, or had a bowel movement.

In an embodiment, the software program may be included as part of an integrated software application for a mobile device. In another embodiment, the system may include at least one subsequent sensing device (e.g. an additional audio sensing or recording device) which can capture data sets and then send information to one or more mobile devices from a remote location. An additional sensing device may be a mobile phone or a wearable device which is configured via integrative software to sense, identify, classify, and interpret data, then transmit the data wirelessly to an output device. In other embodiments, the software may be utilized for multiple end terminal user applications.

In various embodiments, the instant disclosure contemplates product application variations that may incorporate partial data sets that may be applied to any living creature having discernable vocalizations.

### Processing Examples

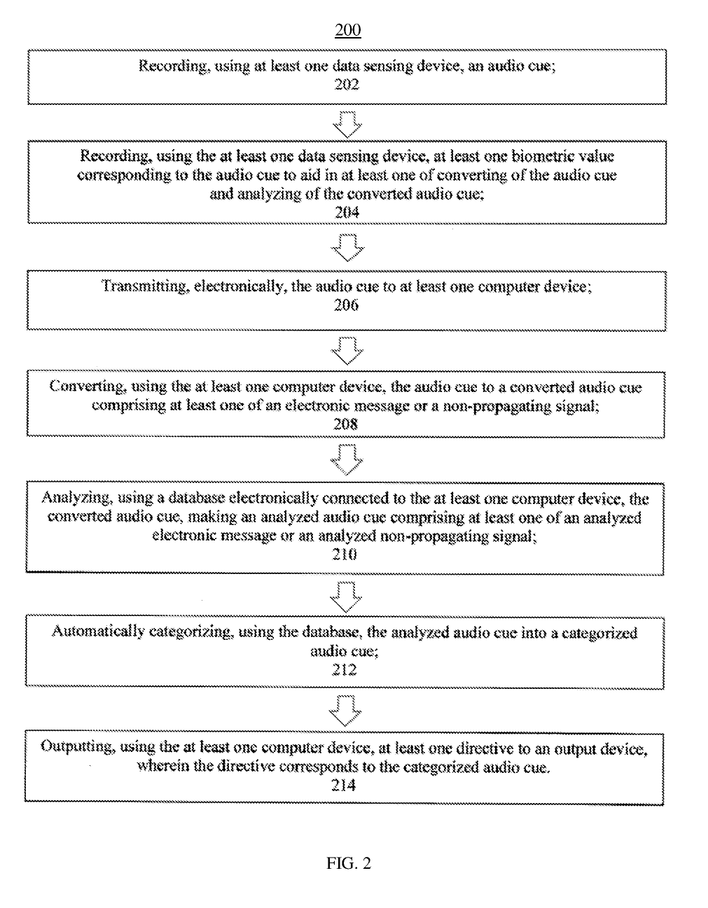

FIG. 2 is a process flowchart corresponding to method (200) for translating baby language into clear communicable word forms, in accordance with various embodiments of the disclosure. As seen in FIG. 2, method (200) comprises recording, using at least one data sensing device, an audio cue (Operation 202). The method further includes recording, using the at least one data sensing device, at least one biometric value corresponding to the audio cue to aid in at least one of converting of the audio cue and analyzing of the converted audio cue (Operation 204). The method continues with transmitting, electronically, the audio cue to at least one computer device (Operation 206). Next, converting, using the at least one computer device, the audio cue to a converted audio cue comprising at least one of an electronic message or a non-propagating signal (Operation 208). In an embodiment, the method subsequently includes analyzing, using a database electronically connected to the at least one computer device, the converted audio cue, making an analyzed audio cue comprising at least one of an analyzed electronic message or an analyzed non-propagating signal (Operation 210). Automatically categorizing, using the database, the analyzed audio cue into a categorized audio cue (Operation 212) is an additional action, and then the method provides for outputting, using the at least one computer device, at least one directive to an output device, wherein the directive corresponds to the categorized audio cue (Operation 214). In some embodiments, the at least one directive is a translation, diagnostic information, or suggested behavior information.

### Development of the Disclosed Algorithm

The development of a system for translating infant vocalizations and biometric data into meaningful, actionable information has been studied extensively. Work carried out towards this goal encompasses, for example, the simulation of relevant data and the development and evaluation of a neural network-based machine learning model. It is assumed that feature data is randomly distributed about mean values given for each label. It has been found that the relevancy of each feature of the generated data can be assessed by training the model on subsets of the input feature set, constituting a powerful tool for future development work. The various embodiments of this disclosure and the development work that we have carried out are based upon various assumptions.

### Assumptions

In order to carry out our assessments, a number of assumptions were made to enable and facilitate the analysis. These include: (1) That randomly generated feature data will offer a sufficiently realistic dataset for methodology development; (2) Feature data is well approximated by a random normal distribution of values; (3) Categorical Dunstan Baby Language (DBL) classification data can be used as a feature as well as, raw or processed audio data; (4) DBL classifications used as input is 75% accurate, and the distribution of inaccurate classifications is irrelevant; and (5) Standard deviation numbers for each feature that are a few percent of the mean of the label-wise given feature means can be used.

### Key Features of New Model

The disclosed embodiments are based upon the premise of developing a system by which baby vocalizations and biometric data can be collected and used to classify baby needs. This work builds upon a prior machine learning implementation of DBL classification, which maps particular baby vocalizations to particular baby needs. By combining this auditory data from DBL with additional relevant biometric data, the system disclosed herein attains greater accuracy than the existing audio-only model (which was only about 75% accurate). The specific features that will be incorporated into the model are heart rate variability (HRV), electrodermal activity (EDA), respiratory rate (RR), and ambient temperature (TEMP).

Prior to training the model on actual data, datasets were randomly generated assuming random normal distribution for numerical data and assuming a 75% accuracy for categorical DBL classifications. The model was then trained on these randomly generated data sets, using every combination of features to determine how responsive it is to the features individually and to feature interactions.

### Data Generation

The numerical data distributions were based on label-wise average values and standard deviation values and were adjusted until the model converged and were used in a label-agnostic fashion. The assumptions of normality and of the particular standard deviation values were initially made for ease of implementation and have been perfected over the data collection period Further, the inherent randomness of the datasets generated guarantees that no hidden patterns in the data can be discovered by the model which is one of the core strengths of the disclosed machine learning model and the various disclosed embodiments of the baby language translation system. Rather, the predictions made by the disclosed machine learning model trained on random data are more seemingly deterministic and causal in nature and speak more to the model's ability to separate out classes based on where they logically would be based on the input statistical information. Additionally, using label-agnostic standard deviation information simplifies the analysis, though one would expect feature variability to vary greatly between differently labeled examples.

Categorical DBL classification data was generated by assuming that, for a given label, the corresponding vocalization would occur 75% of the time. The feature values for the remaining 25% of values were assigned numbers and were selected such that the probabilities of each feature value occurring summed to unity for each label. The particular distribution of misclassifications, however, could contribute to the predictive power of our model, so deciding these values randomly again limits the insight the model gleans from a particular feature.

The disclosed model was based on 200 examples that were generated for the dataset in anticipation of the expected volume of data collection.

### Disclosed Machine Learning Model

The machine learning model of the disclosed embodiments is a neural network-based classifier. As one of ordinary skill in the art knows, a neural network-based classifier uses hidden layers of nodes subjected to an activation function in order to achieve nonlinearity (without feature crosses). The neural network of the instant disclosure uses two hidden layers composed of 10 nodes each. Adagrad optimization, an adaptive form of Stochastic Gradient Descent (SGD) was used to train the model with a base learning rate of 0.1. The ReLU activation function was used.

Since the model was trained on the training dataset all at once before being evaluated using the test set, this methodology exemplifies offline learning. The model was evaluated for its accuracy, though further insights are gained by generating a confusion matrix or by plotting training and test loss against iteration. A confusion matrix is generated to test the data set when the volume of data being utilized makes this process feasible.

### Initial Model Sample Results

Ranking the features by their importance as measured by the accuracy of our model trained on only that feature yielded the following ranking: HRV (91.4% accuracy), DBL (75.1%), EDA (72.5%), RR (56.8%), and TEMP (25.0%). In contrast, assessing each pair of features led to the following ranking: HRV/EDA (94.7%), HRV/DBL (93.5%), EDA/DBL (93.4%), HRV/RR (77.0%), RR/DBL (76.7%), EDA/RR (75.0%), EDA/TEMP (74.9%), HRV/TEMP (72.2%), TEMP/DBL (68.9%), and RR/TEMP (68.5%).

These results are well-explained by the given averages and assumed standard deviation multipliers as shown in TABLE 1 below. HRV has the highest accuracy for a single feature, with other features significantly less predictive, while the three most predictive feature pairs (the three combinations of HRV, EDA and DBL) are clustered much more closely. This is because the HRV data has unique average values but a higher standard deviation (calculated by multiplying the multiplier by the mean of the average values) than EDA, relative to the range. So, HRV alone does a better job of differentiating between our labels than EDA alone, but only because EDA has the same average value for the ‘sleepiness’ and ‘hunger’ labels. Combining EDA with DBL, which can differentiate between ‘burp’ and ‘hunger’, resolves the ambiguity and allows the model to benefit from the greater precision of DBL/EDA data in comparison to either individual feature.

TABLE 1

| | | | | | Label- | | |
|---|---|---|---|---|---|---|---|
| | | | | | wise | SD | |
| FEATURE | Discomfort | Burp | Hunger | Sleepiness | Average | Multiplier | SD |
| Heartrate | 7 | 6 | 4 | 5 | 5.5 | 0.02 | 0.11 |
| Variation | | | | | | | |
| (HRV) | | | | | | | |
| Electrodermal | 40 | 21 | 5 | 5 | 17.75 | 0.04 | 0.71 |
| Activity | | | | | | | |
| (EDA) | | | | | | | |
| Respiratory | 30 | 25 | 20 | 20 | 23.75 | 0.04 | 0.95 |
| Rate (RR) | | | | | | | |
| Ambient | 75 | 73 | 71 | 71 | 72.5 | 0.03 | 2.175 |
| Temperature | | | | | | | |
| (TEMP) | | | | | | | |

TEMP was a poor predictor. This is because its average values were large and close together, which caused the standard deviation to exceed the separation between average ambient temperature values, leading to a large degree of overlap in the temperature data.

### Model Expected Results

The results presented above provide a lower bound on the accuracy of our model, and produced equivalent or better precision in real data, since subtle patterns occurring in the data can be leveraged by the neural network to achieve greater accuracy and no such patterns exist in random data, inherently. Additionally, features whose standard deviation is large compared to the separation between adjacent values are less predictive.

### Conclusion Re: Disclosed Algorithm

The instant disclosure teaches a methodology by which we can determine a model's sensitivity to various features, given average and standard deviation values for those features, as detailed herein.

The disclosed machine learning model includes aspects of each of the following: Simple linear model—Models output classification as a linear function of input features, iteratively finds weights and bias using some form of optimization. Extreme learning machines (ELM)—Commonly: Single Layer Feedforward Networks (SLFNs); Trained much more quickly than back propagation. K-Nearest Neighbors—Simplest ML algorithm; Instance-based: performs no explicit generalization; Lazy: generalization delayed until query. Naïve Bayes—Simple, easy-to-train model based on an assumption of independent features. Decision Tree Learning—“white-box” model that “learns” a decision tree for classifying data based on input features. Support Vector Machines—Focus on finding “maximum-margin hyperplane” separating the classes. Hierarchical classification—Deals with “hierarchically labelled data.”

Collection and analysis of stringently accurate data is fundamental to generating a model that can make accurate predictions.

### CONCLUSION

Different examples and aspects of the systems and methods are disclosed herein that include a variety of components, features, and functionality. It should be understood that the various examples and aspects of the systems and methods disclosed herein may include any of the components, features, and functionality of any of the other examples and aspects of the systems and methods disclosed herein in any combination, and all of such possibilities are intended to be within the spirit and scope of the present disclosure.

Many modifications and other examples of the disclosure set forth herein will come to mind to one of ordinary skill in the art to which the disclosure pertains having the benefit of the teachings presented in the foregoing descriptions and the associated drawings.

What is claimed is:

1. A baby language translation system comprising:
a database comprising at least one software program;
a computer device, wherein the computer device is configured to receive at least one audio cue from an infant and analyze the audio cue using the at least one software program; and
at least one output device configured to connect wirelessly to the computer device;
wherein the database is configured to store a known data set;
wherein the at least one software program comprises an algorithm configured to interact with the database; and
wherein the computer device is configured to recognize the at least one audio cue and use the at least one software program to translate the at least one audio cue and output a translation of the at least one audio cue to the at least one output device.

2. The baby language translation system of claim 1, further comprising a data sensing device comprising an audio sensing device and a recording device,
wherein the recording device is configured to record at least one type of biometric data, wherein the biometric data comprises at least one of a respiration rate (RR), a heart rate variability (HRV), electrodermal activity (EDA), an ambient temperature (TEMP), or a movement feature.

3. The baby language translation system of claim 2, wherein the recording device is further configured to record the at least one audio cue from the infant and transmit the audio cue to the computer device.

4. The baby language translation system of claim 1, wherein the at least one output device comprises a display configured to provide useful auditory or visual output based upon the translation of the at least one audio cue.

5. The baby language translation system of claim 4, wherein the at least one software program is an integrative software which can be utilized as an application available for mobile or wearable devices.

6. The baby language translation system of claim 1, wherein the algorithm combines auditory data from categorical Dunstan Baby Language (DBL) classification data with biometric data including at least one of respiratory rate (RR), heart rate variability (HRV), electrodermal activity (EDA), ambient temperature (TEMP), or movement.

7. The baby language translation system of claim 6, wherein the known data set that is stored in the database comprises the biometric data.

8. The baby language translation system of claim 2, wherein the data sensing device is further configured to track and record data related to at least one of when the infant last ate, slept, urinated, or pooped.

9. The baby language translation system of claim 1, wherein the algorithm comprises a machine learning model.

10. A baby language translation system comprising at least one software or database programmed to receive a translated or converted audio cue and analyze the translated or converted audio cue based on an algorithm which uses a known data set; and
wherein, in response to receiving and analyzing the translated or converted audio cue, the at least one software or database outputs at least one of a response, command, or information,
wherein the at least one of the response, command, or information diagnoses or otherwise provides useful information with respect to the translated or converted audio cue.

11. The baby language translation system of claim 10, further comprising a recording device or an audio sensing device configured to receive an audio cue of a baby.

12. The baby language translation system of claim 11, further comprising at least one output device wirelessly connected to a computer device, wherein the computer device is electronically connected to the at least one software or database and to the recording device or the audio sensing device, and
wherein the at least one output device is configured to provide useful auditory or visual output based upon the translated or converted audio cue received by the recording device or the audio sensing device.

13. The baby language translation system of claim 10, further comprising at least one sensor configured to record at least one type of biometric data from a baby, wherein the at least one type of biometric data comprises a respiration rate (RR), a heart rate variability (HRV), electrodermal activity (EDA), an ambient temperature (TEMP), or a movement.

14. The baby language translation system of claim 13, further comprising a second sensing device configured to track and record data related to at least one of when the baby last ate, slept, urinated, or pooped.

15. The baby language translation system of claim 10, wherein the algorithm combines auditory data from categorical Dunstan Baby Language (DBL) classification data with biometric data including at least one of respiratory rate (RR), heart rate variability (HRV), electrodermal activity (EDA), ambient temperature (TEMP), or movement to provide an analysis of a received audio cue.

16. The baby language translation system of claim 13, wherein the known data set comprises the biometric data.

17. A method for translating baby language into clear communicable word forms, the method comprising:
recording, using at least one data sensing device, an audio cue;
transmitting, electronically, the audio cue to at least one computer device;
converting, using the at least one computer device, the audio cue to a converted audio cue comprising at least one of an electronic message or a non-propagating signal;
analyzing, using a database electronically connected to the at least one computer device, the converted audio cue, making an analyzed audio cue comprising at least one of an analyzed electronic message or an analyzed non-propagating signal;
recording, using the at least one data sensing device, at least one biometric value corresponding to the audio cue to aid in at least one of the converting of the audio cue and the analyzing of the converted audio cue.
automatically categorizing, using the database, the analyzed audio cue into a categorized audio cue; and
outputting, using the at least one computer device, at least one directive to an output device, wherein the directive corresponds to the categorized audio cue.

18. The method of claim 17 wherein the analyzing and the categorizing are completed using an algorithm and a known data set running on a computer system configured to receive translated audio or other information input from the at least one data sensing device; the computer system further comprising the output device configured to connect wirelessly to and interact with the algorithm and known data set via computer software or code on the database; and
the method further comprising outputting, using the algorithm, the at least one directive to the output device, wherein the at least one directive is a translation, diagnostic information, or suggested behavior information.

19. The method of claim 18 wherein the algorithm combines auditory data from categorical Dunstan Baby Language (DBL) classification data with biometric data including at least one of respiratory rate (RR), heart rate variability (HRV), electrodermal activity (EDA), ambient temperature (TEMP), or movement to provide the analyzed audio cue.

20. The method of claim 19 wherein the algorithm is part of an integrated software of a mobile or wearable device application.
